(19) United States
(12) Patent Application Publication
(10) Pub. No.: US 2018/0126018 A1
(43) Pub. Date: May 10, 2018

(54) LOW-PRESSURE SYSTEM INCLUDING DRY STEAM FOR CLEANING SMOKING ACCESSORIES

(71) Applicant: Eric Talner, San Diego, CA (US)

(72) Inventor: Eric Talner, San Diego, CA (US)

(21) Appl. No.: 15/798,258

(22) Filed: Oct. 30, 2017

(51) Int. Cl. A61L 2/07; B08B 3/10; A61L 2/26; A24F 9/12

(52) U.S. Cl. CPC A61L 2/07; B08B 3/106; A61L 2/26; A24F 9/12; A61L 2202/11; B08B 2230/01; A61L 2202/17; A61L 2202/15; B08B 2203/007

(57) ABSTRACT

A low-pressure system for cleaning items includes a tub, at least one steam head mounted in the tub having a rotatable nozzle configured to direct steam towards the bottom of the tub. The steam generator is in fluid communication with both the first steam head and the second steam head. A valve including a foot pedal mounts in fluid communication between the steam generator and each steam head. The steam generator connects to a water and power source for generating dry steam at a temperature exceeding 300° F. and at a pressure of less than 150 psi. The dry steam maintains a dryness fraction of 0.4-0.9 at all points within the system.

### BACKGROUND OF THE INVENTION

Medical and adult use cannabis has been legalized in many nations across the world. In the United States more than half of the states allow for cultivation distribution and use of cannabis products. Smoking cannabis flowers is the traditional mode of delivery because it enables rapid absorption of bio-active cannabinoids. Smoking cannabis also avoids first pass hepatic metabolism which removes over 90% of the active cannabinoids delivered orally and through the digestive tract.

There are drawbacks with smoking. The heat from the smoke can damage the mouth and throat as well as the lungs. Smoke also includes a number of toxins. Both of these problems can be mitigated through the use of appropriate smoking accessories. Water pipes both cool the smoke and function to filter at least some toxins from the smoke. This enables the consumer to have the benefit of consuming cannabis while mitigating possible health problems associated with smoking marijuana cigarettes, for example.

Other types of smoking accessories can cool the smoke, such as a thermally conductive pipe which draws heat from the smoke, or a pipe with an expansion chamber.

The problem with pipes of all sorts including water pipes is that resin from the smoke cannabis clings to the inside of the pipe. Over time this resin builds up and leaves a black residue. The black residue as an undesirable smell, looks dirty, and is generally unappealing. In a water pipe, the resin residue may provide a substrate for cultures of microbiology that in turn generate additional pungent aromas, and perhaps unhealthful effects.

Most consumers prefer smoking with clean smoking accessories, particularly smoking accessories which deliver a clean, temperature moderated, and healthful experience. Such customers are thus faced with the choice of spending time and effort to clean smoking accessories on a regular basis, to smoke with an unclean smoking accessory, or to not smoke at all.

What is desired is a fast and easy way to clean smoking accessories in an effective and hygienic manner.

### SUMMARY OF THE INVENTION

The present invention includes a system for cleaning smoking accessories. The system includes a tub having a bottom for capturing fluid, a first steam head, and a second steam head. The first steam head mounts in the tub and has a rotatable nozzle configured to direct steam towards the bottom of the tub, in other directions. The second steam head mounts in the tub and has a fixed nozzle configured to direct steam upwards, away from the bottom of the tub. A benefit of the present invention is to sterilize smoking accessories and other objects to inhibit the spread of pathogenic microbiology including viruses, fungi and bacteria. One benefit of having a rotatable nozzle is that the tub itself can be cleaned by the nozzle in addition to smoking accessories.

The invention includes a steam generator in fluid communication with both the first steam head and the second steam head to deliver dry steam at low pressures to the steam heads. Preferably, the pressures are kept below 150 psi to maximize safety of a user of the system. More preferably the pressure is between 14-100 psi. In an alternate embodiment the pressure is variable, and is kept between 14 through 50 psi. The system of the present invention is a low-pressure system that enables the temperature of the steam to clean smoking accessories. The system can also be used to clean residue from other glassware, tableware, ceramics and other pottery.

Preferably the temperature of the dry steam is between 300° F. and 350° F. It is possible to use the present invention in conjunction with steam having a temperature of less than 300° F., or more than 350° F., but the ideal range is between 300° F. and 350° F. The temperature is variable in one aspect of the invention, and fixed in another aspect of the invention.

A pressure regulator, or valve mounts in fluid communication between the steam generator and at least one of the first and second steam heads to regulate steam delivery pressure. Preferably, the pressure regulator, or valve includes a foot-actuated device so that a user can use both hands to articulate an item to be cleaned with respect to the steam heads. Preferably, the item to be cleaned is a smoking accessory.

The steam generator is configured to connect to a water source and to a power source for generating dry steam and delivering the dry steam to at least one of the steam heads. The steam generator has sufficient heating capacity to enable use of both steam heads simultaneously. In this way, two operators of the system can clean smoking accessories, or other item, simultaneously at different, selectively regulated pressures and steam flow rates.

In one embodiment of the invention, the fixed nozzle of the second steam head threadedly attaches to a conduit, the conduit extends vertically from the bottom of the tub and attaches in fluid communication with at least one of said valves.

In another embodiment of the invention, the rotatable nozzle of the first steam head threadedly attaches to a conduit, the conduit extends vertically from the bottom of the tub and attaches in fluid communication with at least one of said valves.

The steam heads can be interchanged to create two, or more, rotatable nozzles in a single system. The steam heads can be interchanged to create two, or more, fixed nozzles in a single system.

The system can be collated as a kit of parts including any combination of components described herein. For example, a kit of parts excluding the tub can be assembled and packaged in accordance with the present invention.

### BRIEF DESCRIPTION OF THE DRAWINGS

FIG. 1 is a diagram of a system in accordance with the present invention.

FIG. 2 is a perspective view of portion of the system of FIG. 1, showing a fixed steam head with a removable nozzle.

FIG. 3 is a perspective view of a portion of the system of FIG. 1 showing a movable steam head with a rotatable nozzle.

FIG. 4 is a perspective view of a valve including a foot pedal.

FIG. 5 is a perspective view of a steam head and nozzle cleaning a smoking accessory.

FIG. 6 is a perspective view of a moveable steam head and nozzle and a smoking accessory.

FIG. 7 is a top perspective view of a nozzle.

FIG. 8 is a bottom perspective view of a nozzle.

FIG. 9 is a hidden line perspective view of a nozzle sleeve covering a single port nozzle.

### DETAILED DESCRIPTION

Dry steam is saturated steam that has been very slightly superheated. It results when water is heated to the boiling point and then vaporized with additional heat. If this steam is then further heated above the saturation point, it becomes superheated steam. In dry steam, all the heat which is present is used to transform the water into steam, therefore no micro drops are present. Dry steam sterilizes surfaces upon contact.

When steam is produced in a boiler or steam generator it usually has some moisture present, which comes from the water from which it is generated. The presence of any such moisture is sufficient to render the steam “wet.” The clean/pure steam generators used in the pharmaceutical industry are designed to produce “dry” steam, a clear, colorless vapor with no entrained moisture.

Steam dryness has a direct effect on the total amount of transferable energy contained within the steam, which affects heating efficiency and quality. The steam dryness fraction is used to quantify the amount of water within steam. If steam contains 10% water by mass, it's said to be 90% dry, or have a dryness fraction of 0.9.

Moisture in steam increases the amount of corrosion. Moisture traveling at high steam velocities erodes valve seats and fittings, a condition known as wiredrawing. It also causes erratic operation of control valves. Pure steam cannot carry any impurities, but water can. These impurities increase scaling of pipework and heating surfaces. Impurities can leave water spots on glass and other items to be cleaned. Accordingly, the use of dry steam yields virtually spotless smoking accessories. This is particularly important when cleaning glass pipes such as clear glass water pipes.

Increasing pressure on a volume of dry steam may cause the saturation point to be met, accordingly, dry steam should have pressure managed so that the saturation point is not reached, and formation of water droplets is avoided.

The present invention relies on a low-pressure system that is temperature stable to enable the maintenance of dry steam for use in cleaning smoking accessories.

Dry steam utilized by the present invention is preferably has a dryness fraction of between 0.4 and 0.9 when produced by the steam generator, and does not fall beyond the range of 0.1 and 0.9 anywhere throughout the system of the present invention.

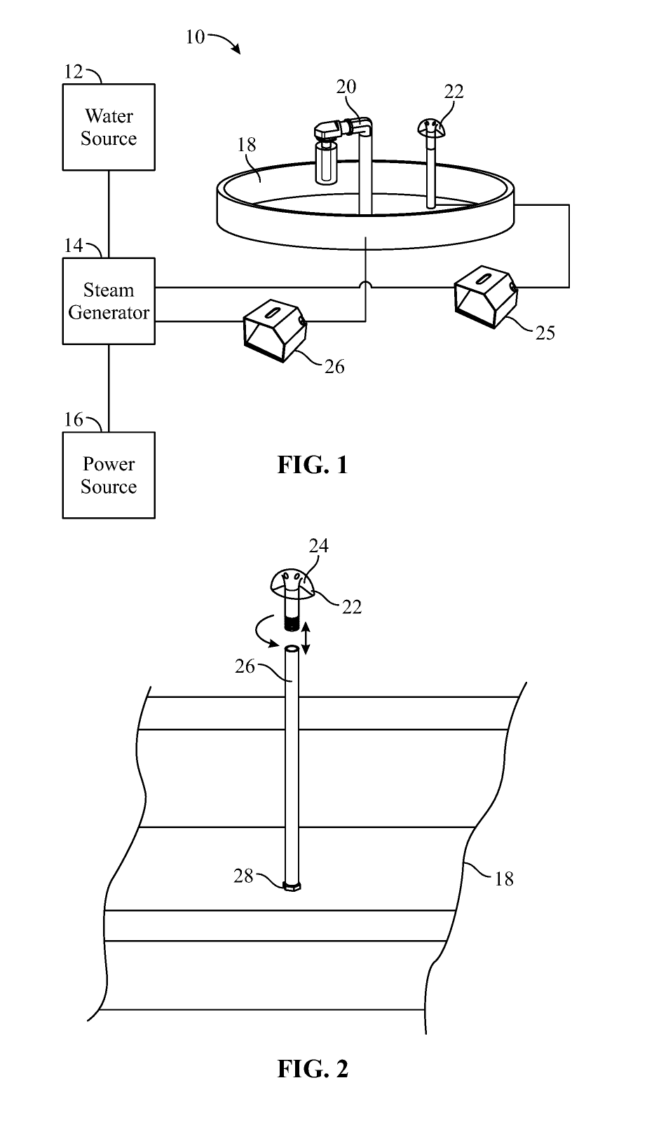

FIG. 1 is a system in accordance with the present invention, generally designated with the reference numeral 10. The system 10 includes a water source 12, steam generator 14, power source 16, at least one pressure regulator 26, a tub 18, and at least one steam head 20.

In a preferred embodiment, two pressure regulators 24 and 25 operatively connect in fluid communication with the respective steam head 22 and 20. The pressure regulators 24 and 25 are a simple valve in one embodiment of the invention.

The pressure regulators 24 and 25 preferably include a foot pedal valve that enables operation of the pressure regulators 24 and 25 by use of a user's foot. The pressure regulators 24 and 25 are connected in fluid communication with the steam generator 14.

The steam generator 14 generates dry steam having a dryness fraction of between 0.4 and 0.9. As steam from the steam generator 14 moves towards the pressure regulators 24 and 25, the dryness fraction and the pressure is maintained. The pressure regulators 24 and 25 have a variable output, and may reduce the dryness fraction. Notwithstanding any reduction of the dryness fraction, the dry steam remains dry, so that no water droplets form within the system 10.

Upon exiting the system 10 via one of the steam heads 20 or 22, the dry steam will cool and release moisture in the form of water droplets. The heat from the dry steam melts resins on any smoking accessory to be cleaned so that the resins change from a solid to a liquid. The moisture released from the dry steam in the form of water droplets carries the liquid resin from the smoking accessory to be cleaned into the tub 18. Accordingly, one mechanism of action in cleaning smoking accessories is due to the combination of heat and condensation to remove liquefied resins from the smoking accessory. Where an internal surface is to be cleaned, the condensation carries away liquefied resins, but it can be appreciated that in the case of a material to be cleaned made of a thermally conductive material, such as glass, the dry steam sprayed on one side can melt resins on the other side. The melted resins can be removed by a dry cloth, without need for direct contact with the dry steam.

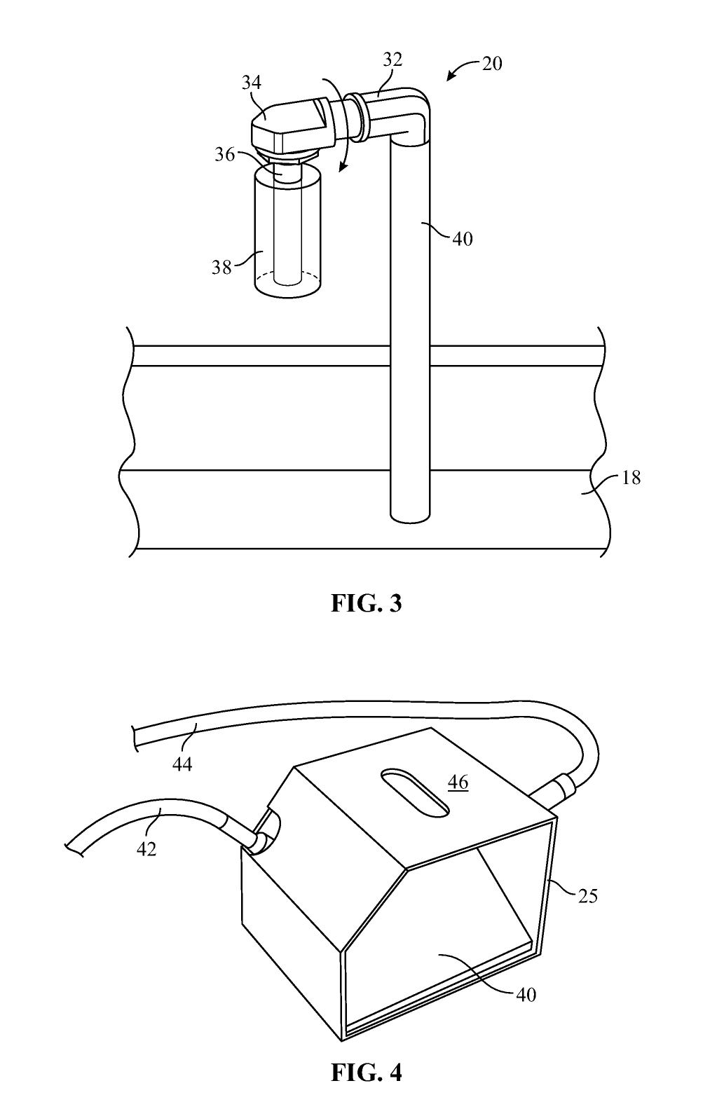

FIG. 2 shows a portion of the tub 18 and the steam head 22. The steam head 22 includes a dome-shaped outlet 24 having a number of steam vents. The steam head 22 threadedly attaches to the tube 26. The tube 26 threat of the mounts to the opening 28, which is formed in the base of the tub 18. The opening 28 enables a fluid connection between the steam head 22 and the dry steam source.

The steam head 22 is fixed in a vertical alignment with respect to the base of the tub 18, which lies in a horizontal plane.

FIG. 3 shows a portion of the tub 18 and the steam head 20. The steam head 20 includes a tube 40, which extends vertically upward from the tub 18. The first elbow 32 mounts on a distal end of the tube 40. A second elbow 34 connects with the first elbow 32 and rotates with respect to the first elbow 32. The second elbow 34 includes a nozzle 36 for directing dry steam in a direction towards the tub 18 during normal operation. The nozzle 36 is shrouded by a sleeve 38.

The sleeve 38 is manufactured from a heat resistant and semi-flexible material to press-fit over the nozzle 36. Preferably the sleeve 38 is made from silicon. The sleeve 38 protects an operator from condensed water that may splash from the region surrounding the nozzle 36 during use. A sleeve 38 also guides steam towards the object to be cleaned. The sleeve 38 may seal against the smoking accessory to direct dry steam into a channel, bowl, stein or other portion of the smoking accessory. Accordingly, the inside of a pipe, such as a mouthpiece, carburetor, bowl, stein or other portion of the pipe may be readily cleaned without misdirection of steam, water vapor, or condensed water.

FIG. 4 shows a pressure regulator 25. The pressure regulator 26 includes an input conduit 42 configured for fluid communication with a steam generator. The pressure regulator 25 includes an output conduit 44 capable of connection to a steam head. The pressure regulator 26 further includes a foot pedal 40. The foot pedal 40 selectively regulates pressure in the output conduit 44. The use of a foot actuated pressure regulator is a hands-free component of the system of the present invention, which enables an operator to use both hands when cleaning smoking accessory.

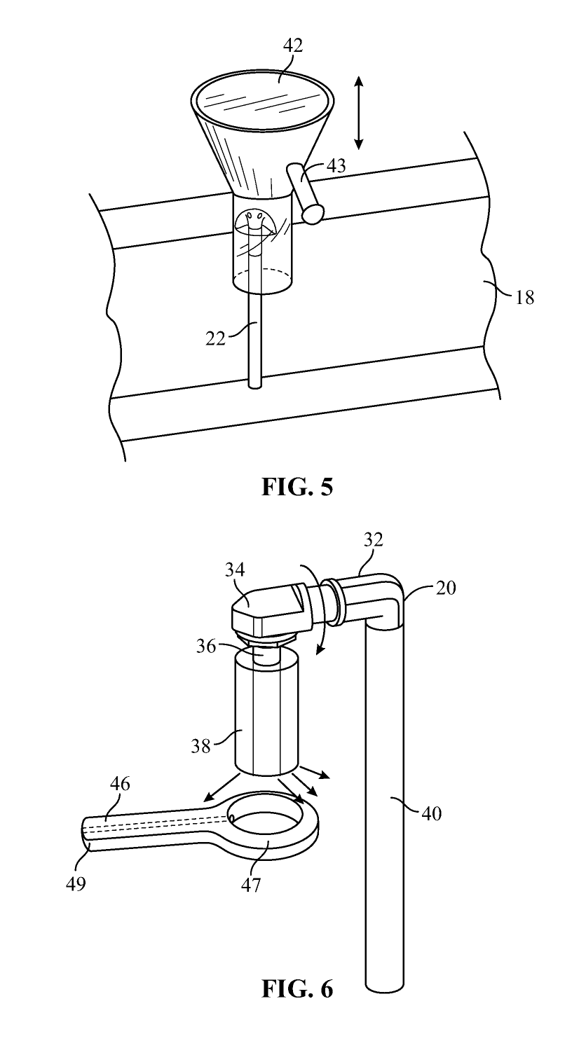

FIG. 5 shows a portion of the tub 18 and the steam head 22 inserted into the mouth of a water pipe 42. A water pipe 42 includes a stein 43 having a diameter of between 14-18 mm. The steam head 22 is sized to be capable of insertion into the stein 43. Thus, the steam head 22 including any nozzle, has a maximum diameter of less than 14 to 18 mm to enable the stein 43 to be optimally be cleaned according to one embodiment of the invention.

FIG. 6 shows the tube 40, the steam head 20, the first elbow 32, the second elbow 34, the nozzle 36 and the sleeve 38 directing dry steam at a pipe 46. The pipe 46 includes a bowl 47. In operation the ball 47 seals to the sleeve 38 to maximize cleaning process, and to inhibit loss of the dry steam. The pipe 46 further includes a mouthpiece 49. During the second stage in the cleaning process mouthpiece 49 seals within the sleeve 38 and against the nozzle 36 to clean inside of the pipe 46. Accordingly, the sleeve 38 has a dual purpose of selectively sealing over a bowl 47 as well as ceiling over a mouthpiece 49, while improving safety for a user, and inhibiting loss of dry steam.

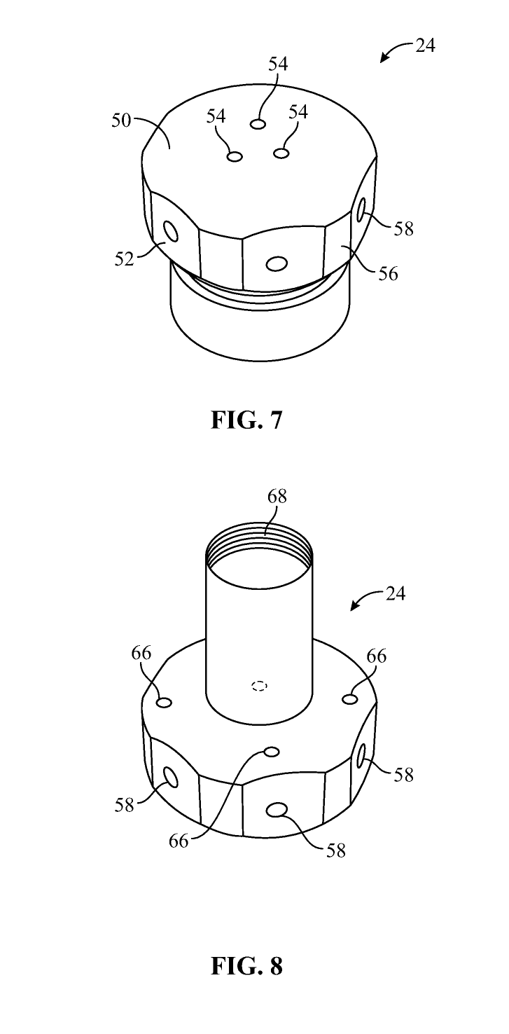

FIG. 7 is a nozzle 24. The nozzle 24 has a curved surface 50 that is convex in shape. The surface 50 defines a number of distal steam ports 54. As shown three steam ports 54 are defined by the surface 50, but this number can vary. The steam ports 54 have a diameter of between 0.5-1.5 mm in diameter, and are preferably 1 mm in diameter. The nozzle 24 also has at least one lateral steam port 58, defined on a lateral periphery of the nozzle 24.

FIG. 8 shows the nozzle 24 having a number of proximal steam ports 66 facing in a direction distal the steam ports 54 of FIG. 7. The nozzle 24 is configured with threads 68 to enable attachment of the nozzle 24 to a steam head.

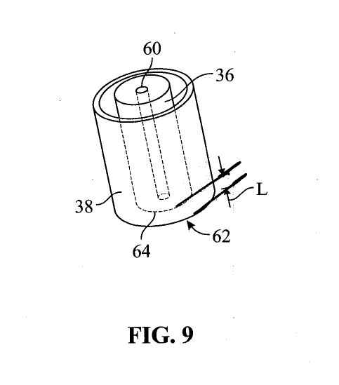

FIG. 9 is a nozzle 36 shrouded in a sleeve 38. The sleeve 38 has an end that extends a distance “L” from a distal end 64 of the nozzle 36. Preferably the distance L is less than one inch to optimally mate with either the bowl of a pipe or the mouthpiece of pipe. In this embodiment, the nozzle 36 includes a single opening 60, which is defined by a cylindrically shaped exterior. The opening has a diameter of between 4-5 mm to inhibit over pressurization and condensation of the dry steam within the nozzle 36.

While the present invention is described by way of examples of various embodiments, the true scope of the invention is defined by the appended claims.

I claim:

1. A low-pressure system for cleaning items comprising:
a tub having a bottom for capturing fluid;
at least one steam head mounted in the tub;
a steam generator in fluid communication with both the at least one steam head;
a first foot pedal mounted in fluid communication between the steam generator and the at least one steam head; and
the steam generator being configured to connect to a water source and to a power source for generating dry steam at a temperature exceeding 300° F. and at a pressure of less than 150 psi,
wherein the steam generator produces steam having a dryness fraction of 0.4-0.9.

2. The system as set forth in claim 1, wherein the at least one steam head includes:
a first steam head mounted in the tub and having a rotatable nozzle configured to direct steam towards the bottom of the tub;
a second steam head mounted in the tub and having a fixed nozzle configured to direct steam upwards, away from the bottom of the tub.

3. The system as set forth in claim 2, where the first foot pedal is in fluid communication with the first steam head, and a second foot pedal is in fluid communication with the second steam head to enable more than one operator to use the system.

4. The system as set forth in claim 3, wherein the dryness fraction of dry steam anywhere in the system stays within the range of 0.1-0.9.

5. The system as set forth in claim 4, wherein the pressure in the system does not exceed 50 psi anywhere in the system.

6. The system a set forth in claim 1, wherein the steam head includes a nozzle with a single port having a diameter of between 3-5 mm.

7. The system as set forth in claim 6, wherein the steam head includes a sleeve press-fit around the nozzle, the nozzle has a distal end, the sleeve extends less than 1 inch beyond the distal end of the nozzle.

8. A low-pressure system for cleaning resin from smoking accessories comprising:
a tub having a bottom for capturing fluid;
at least one steam head mounted in the tub;
a steam generator in fluid communication with both the at least one steam head;
a first foot pedal mounted in fluid communication between the steam generator and the at least one steam head; and
the steam generator being configured to connect to a water source and to a power source for generating dry steam at a temperature of approximately 350° F. and at a pressure of less than 50 psi,
wherein the steam generator produces steam having a dryness fraction of 0.4-0.9.

9. The system as set forth in claim 8, wherein the at least one steam head includes:
a first steam head mounted in the tub and having a rotatable nozzle configured to direct steam towards the bottom of the tub;
a second steam head mounted in the tub and having a fixed nozzle configured to direct steam upwards, away from the bottom of the tub.

10. The system as set forth in claim 9, where the first foot pedal is in fluid communication with the first steam head, and a second foot pedal is in fluid communication with the second steam head to enable more than one operator to use the system.

11. The system as set forth in claim 10, wherein the dryness fraction of dry steam anywhere in the system stays within the range of 0.1-0.9.

12. The system as set forth in claim 11, wherein the pressure in the system does not exceed 50 psi anywhere in the system.

13. The system a set forth in claim 8, wherein the steam head includes a nozzle with a single port having a diameter of between 3-5 mm.

14. The system as set forth in claim 13, wherein the steam head includes a sleeve press-fit around the nozzle, the nozzle has a distal end, the sleeve extends less than 1 inch beyond the distal end of the nozzle.

15. A method of using dry steam to melt and remove resin from a cannabis pipe, comprising:
providing a pipe tarnished with cannabis resin; and
directing dry steam having a dryness fraction of 0.4-0.9 on to the pipe to melt the resin, condense a portion of the dry steam into water droplets, and to enable the water droplets to assist in removal of the melted resin.

16. The method of claim 15, wherein the pipe is a water pipe including a mouth and a stem, and the method includes sequentially directing dry steam both through the mouth and the stem.

17. The method of claim 15 further comprising dispensing the dry steam through a nozzle having a distal end and a silicon sleeve surrounding the nozzle and extending less than one inch from the distal end.

18. The method of claim 17, wherein the pipe includes a mouthpiece and a bowl, and the method includes sealing the mouth piece and bowl against the silicon sleeve.

19. The method of claim 17, wherein the pipe includes a mouthpiece and a bowl, the method includes sealing the mouth piece against both the distal end of the nozzle and the silicon sleeve.
